(19) United States
(12) Patent Application Publication
(10) Pub. No.: US 2024/0342332 A1
(43) Pub. Date: Oct. 17, 2024

(54) STERILE PACKAGE OPENING SYSTEM AND METHODS

(71) Applicant: Benjamin S. Paras, Tucson, AZ (US)

(72) Inventor: Benjamin S. Paras, Tucson, AZ (US)

(21) Appl. No.: 18/750,371

(22) Filed: Jun. 21, 2024

Related U.S. Application Data
(63) Continuation-in-part of application No. 17/070,770, filed on Oct. 14, 2020.
Application No. 17/070,770 is a continuation of application No. 15/957,744, filed on Apr. 19, 2018, now Pat. No. 10,881,754.
(60) Provisional application No. 62/487,324, filed on Apr. 19, 2017.

(51) Int. Cl. A61L 2/23; A61L 2/18; B65D 75/30; B65D 75/58; B65D 83/00

(52) U.S. Cl. CPC A61L 2/23; A61L 2/186; B65D 75/30; B65D 75/5805; B65D 83/0038; A61L 2202/18

(57) ABSTRACT

A system and method to open an antiseptic package includes a bacteria-proof packaging container having a first portion and a second portion that form an interior compartment. A medical implement is positioned within the interior compartment. The packaging container has at least one pocket to receive an end of a tool. The tool is manipulable to separate the first portion from the second portion. A portion of packaging container is removable from the antiseptic wipe to expose the medical implement while an unexposed end of the medical implement is graspable by a user through the packaging container.

### CROSS REFERENCE TO RELATED APPLICATIONS

This application is a continuation-in-part of U.S. application Ser. No. 17/070,770 filed Oct. 14, 2020, which itself is a continuation of U.S. patent application Ser. No. 15/957,744 filed Apr. 19, 2018, now U.S. Pat. No. 10,881,754, granted Jan. 5, 2021, which claims benefit of U.S. Provisional Application Ser. No. 62/487,324 filed Apr. 19, 2017, titled “Disposable Antiseptic Wipe Apparatus,” the entire disclosures of which are incorporated herein by reference.

### FIELD OF THE DISCLOSURE

The present disclosure is generally related to antiseptic packaging and more particularly is related to a system and method to maintain sterility of a medical instrument in an antiseptic package while opening the package.

### BACKGROUND OF THE DISCLOSURE

Sterile medical implements are conventionally stored in sterile packaging. For example, antiseptic wipes may be stored in a sealed foil packet. Other sterile packaging may be similar to “peel pouches.” The peel pouches are formed from two portions of packaging that are typically adhesively attached along the edges to form an interior compartment to hold sterile medical implements. To access a medical implement inside one of these sterile packages, a user is required to tear a portion of the package or grasp the edges of the package and pull portions of the package apart to separate them.

FIGS. 1-3 are illustrations of antiseptic wipes 10 in accordance with the prior art. Specifically, FIG. 1 illustrates a conventional antiseptic wipe 10 showing the fabric material 12 within the packaging 14 and FIG. 2 illustrates the antiseptic wipe 10 in a state where the packaging 14 has been opened to allow contact to the antiseptic-infused fabric material 12. FIG. 3 depicts the antiseptic-infused fabric material 12 after removal from the packaging 14. Commonly, the antiseptic-infused fabric material 12 is a single sheet of material which is folded into layers prior to being placed within the packaging, which can then be unfolded after removal from the packaging 14, as shown in FIG. 3. When antiseptic wipes are used, the fabric material is removed from the packaging, optionally unfolded, and then rubbed around the injection area of the patient or the IV port within the patient to clean the selected area with the antiseptic material infused in the fabric material. The contact between the antiseptic-infused fabric material 12 and the selected area on the patient is generally successful in reducing the possibility of infection or sepsis during injection or through the IV port.

In another example, an antiseptic wipe infused with an antiseptic material may be inside a foil-lined packet. When antiseptic wipes are used, the fabric material is typically removed from the packaging, optionally unfolded, and then rubbed around the injection area of the patient or the IV port within the patient to clean the selected area with the antiseptic material infused in the fabric material. The contact between the antiseptic-infused fabric material and the selected area on the patient is generally successful in reducing the possibility of infection or sepsis during injection or through the IV port.

While the antiseptic material rarely causes irritation to the patient's skin, due to the infrequent use of the antiseptic wipe on the patient's skin, the antiseptic material can cause significant irritations to the fingers of the nurse, doctor, or other medical professional using the antiseptic wipe numerous times a day. In fact, it is not uncommon for a medical professional to use an antiseptic wipe 30-50 times daily. With this repeated use, the fingers and thumb of the user are continually and repeatedly exposed to the antiseptic material, which leads to their fingers and thumb becoming sore, having cracked, dry skin which bleeds, and eventually leads to a decreasing of the fingerprint ridges on their skin. The effect on the user's fingers is painful and it leads to negative aesthetic issues on the user's fingers. Some users have attempted to solve this problem by wearing disposable medical gloves (latex or otherwise) when they need to use an antiseptic wipe, but using 30-50 gloves each day is wasteful, inefficient, and expensive. A standard disposable latex glove costs a medical facility approximately $0.06 each. Over a one-year period, a medical professional who uses 50 gloves a day will cost the medical facility approximately $750. There are also significant negative environmental consequences of using so many non-biodegradable gloves.

FIGS. 4-5 are illustrations of peel pouch 20 in accordance with the prior art. Specifically, FIG. 4 illustrates a conventional peel pouch 20 showing a first portion 22 and a second portion 24 of the packaging and a medical implement 26 within an internal chamber 28 created between the two portions of the packaging 20. FIG. 5 illustrates a peel pouch 20 partially opened by a medical practitioner by grasping a first portion 22 in one hand, a second portion 24 in another hand, and separating the first portion 22 from the second portion 24 along a separation structure 30 to expose the medical implement 26. The medical implement 26 may be any type of implement contained in sterile packaging, such as a swab, a wipe, a syringe, or other surgical instrument.

The use of a conventional antiseptic peel pouch 20 is also not without shortcomings. In a busy surgical environment, a medical practitioner or medical professional may have only one hand available to grasp one of the edges, and so he or she may have difficulty holding and opening the package. A user may even use their teeth to grasp an edge to separate the packaging and access the medical implement inside. In this type of situation, a sterile implement may come into contact with a non-sterile environment upon opening its package and before it is used on a patient.

Further, a medical professional opening the package may need to retain a grasp on the implement inside the sterile packaging to prepare it for use on the patient while maintaining its sterility. For example, medical syringes typically come inside sterile packaging. The syringe within the packaging compartment is accessed when a medical professional separates one portion of the package from another. Once the syringe is accessible, the medical professional must further manipulate the syringe by pulling or pushing and twisting the syringe to remove the cap covering a needle or to attach a needle to the syringe. Firmly holding onto the medical implement while it remains partially in the sterile packaging maintains the sterility of the implement during this manipulation, but holding the implement inside the packaging may be difficult to accomplish after separating the packaging portions to access the implement.

Thus, a heretofore unaddressed need exists in the industry to address the aforementioned deficiencies and inadequacies.

### SUMMARY OF THE DISCLOSURE

Embodiments of the present disclosure provide a system and method for opening a sterile package. Briefly described, in architecture, one embodiment of the system, among others, can be implemented as follows.

A system for opening a package while maintaining sterility of a medical implement within a bacteria-proof packaging container has an interior compartment. A first portion of the bacteria-proof packaging container is separatable from a second portion of the bacteria-proof packaging container. A medical implement is positioned within the interior compartment. At least one pocket is positioned proximate to a top edge of the bacteria-proof packaging container. A tool is configured for opening the bacteria-proof packaging container. The tool has at least one end sized to fit within the at least one pocket. A first end of the tool is mounted substantially perpendicular to a second end of the tool, and the tool is controllable to separate the first portion from the second portion. Further, the bacteria-proof packaging container has a vertical separation structure positioned between the first portion and the second portion, and axially along the bacteria-proof packaging container. Pulling the at least one pocket by the tool causes the bacteria-proof packaging container to open along the vertical separation structure to separate the first portion from the second portion. The tool has a first side and a second side. The first and second sides are positionable on opposite sides of the medical implement. A member joins the first side to the second side. First and second pockets are further positioned on the first and second portions, respectively. Each of the first and second pockets are positioned proximate to the top edge of the bacteria-proof packaging container. The first and second pockets are configured to receive the first and second sides of the tool, respectively. When the first side and the second side are positioned in the first and second pockets, respectively, the first side and second side are moveable away from each other to separate the first portion from the second portion. The tool is sized to separate a portion of the bacteria-proof packaging container to expose the medical implement in the interior compartment.

The present disclosure can also be viewed as a tool with a member that is a housing with a first contact structure on the housing. The first contact structure is sized to accommodate a first finger. An insert is slideably positionable relative to the housing and operably connected to the first side and the second side. The insert has a second contact structure sized to accommodate a second finger. A bias mechanism is positioned between the insert and the housing. The bias mechanism biases the first contact structure away from the second contact structure. Moving the insert towards the housing causes the first side and the second side to move away from each other.

The present disclosure can also be viewed as providing a packaging container for maintaining sterility of a medical implement within the package during unsealing. A first portion of the bacteria-proof packaging container is sealed to and separatable from a second portion of the bacteria-proof packaging container. The seal between the first portion and second portion creates an interior compartment between the first portion and the second portion. A medical implement is positioned within the interior compartment. At least one pocket is positioned proximate to a top edge of the bacteria-proof packaging container. The at least one pocket is formed from an overfolded section connected along lateral edges thereof to the first portion. The at least one pocket is located interior of the overfolded section and the lateral edges. Further, the at least one pocket has first and second pockets positioned on the first and second portions, respectively. Each of the first and second pockets are positioned proximate to the top edge of the bacteria-proof packaging container. The first and second pockets are configured to receive the first and second sides of the tool, respectively. When the first side and the second side are positioned in the first and second pockets, respectively, the first side and second side are moveable away from each other to separate the first portion from the second portion.

The present disclosure can also be viewed as providing methods of opening a package while maintaining sterility of a medical implement within the package. In this regard, one method, among others, can be broadly summarized by the following steps: positioning a tool over the medical implement within an interior compartment in a bacteria-resistant packaging container; inserting a first end of the tool into a pocket proximate to a top edge of the bacteria-resistant packaging container; and actuating the tool to move to a second end of the tool mounted substantially perpendicularly to the first end of the tool toward the item, thereby separating a first portion of the bacteria-resistant packaging container from a second portion of the bacteria-resistant packaging container.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1-3 are illustrations of antiseptic wipes in accordance with the prior art.

FIGS. 4-5 are illustrations of peel pouches in accordance with the prior art.

FIGS. 6-8 are illustrations of a bacteria-proof packaging container, in accordance with embodiments of the present disclosure.

FIGS. 9A-9C are illustrations of a system having a tool for opening a bacteria-proof packaging container, in accordance with embodiments of the present disclosure.

FIGS. 10A-10B are illustrations of a bacteria-proof packaging container, in accordance with embodiments of the present disclosure.

FIG. 11 is an illustration of a bacteria-proof packaging container, in accordance with embodiments of the present disclosure.

FIGS. 12A and 12B are illustrations of a bacteria-proof packaging container, in accordance with embodiments of the present disclosure.

FIG. 13 is an illustration of a bacteria-proof packaging container, in accordance with embodiments of the present disclosure.

FIG. 14 is an illustration of a bacteria-proof packaging container, in accordance with embodiments of the present disclosure.

FIG. 15 is an illustration of a bacteria-proof packaging container, in accordance with embodiments of the present disclosure.

FIG. 16 is an illustration of a bacteria-proof packaging container, in accordance with embodiments of the present disclosure.

FIG. 17 is an illustration of a bacteria-proof packaging container and a system having a tool interacting with the bacteria-proof packaging container, in accordance with embodiments of the present disclosure.

FIG. 18 is an illustration of a bacteria-proof packaging container and a tool interacting with the bacteria-proof packaging container, in accordance with embodiments of the present disclosure.

FIG. 19 is an illustration of a bacteria-proof packaging container and a tool interacting with the bacteria-proof packaging container in accordance with embodiments of the present disclosure.

FIG. 20 is an illustration of a bacteria-proof packaging container, in accordance with embodiments of the present disclosure.

FIGS. 21-22 are illustrations of a tool, in accordance with embodiments of the present disclosure.

FIG. 23 is an illustration of a bacteria-proof packaging container and a tool interacting with the bacteria-proof packaging container, in accordance with embodiments of the present disclosure.

FIGS. 24A-24C are illustrations of a tool, in accordance with embodiments of the present disclosure.

FIG. 25 is an illustration of a tool interacting with a bacteria-proof packaging container, in accordance with embodiments of the present disclosure.

FIGS. 26A-26B are illustrations of a tool, in accordance with embodiments of the present disclosure.

FIG. 27 is an illustration of a tool interacting with a bacteria-proof packaging container, in accordance with embodiments of the present disclosure.

FIGS. 28A-28C are illustrations of a tool interacting with a bacteria-proof packaging container, in accordance with embodiments of the present disclosure.

FIGS. 29-30 are illustrations of a tool, in accordance with embodiments of the present disclosure.

FIGS. 31A-31B are illustrations of a tool interacting with a bacteria-proof packaging container, in accordance with embodiments of the present disclosure.

FIG. 32 is an illustration of a tool interacting with a bacteria-proof packaging container, in accordance with embodiments of the present disclosure.

FIG. 33 is an illustration of a medical professional using a tool to open a bacteria-proof packaging container and use a medical implement on a patient, in accordance with embodiments of the present disclosure.

FIG. 34 is a diagrammatical flowchart illustrating a method of opening a sterile package using a tool, in accordance with embodiments of the present disclosure.

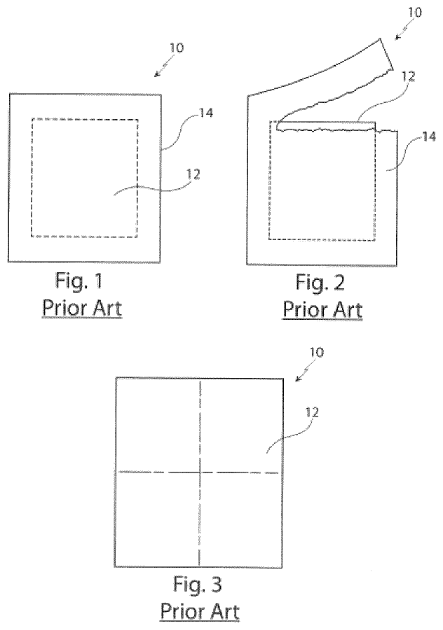

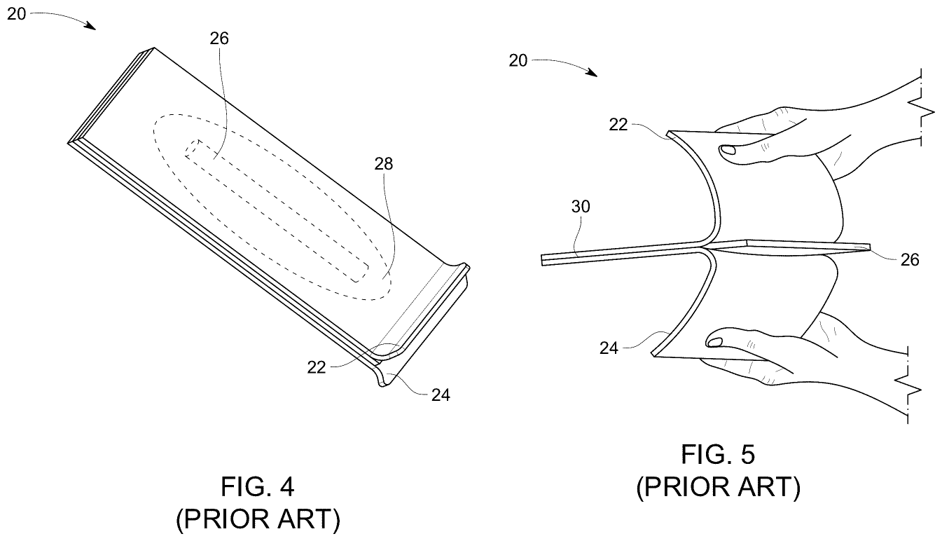

### DETAILED DESCRIPTION

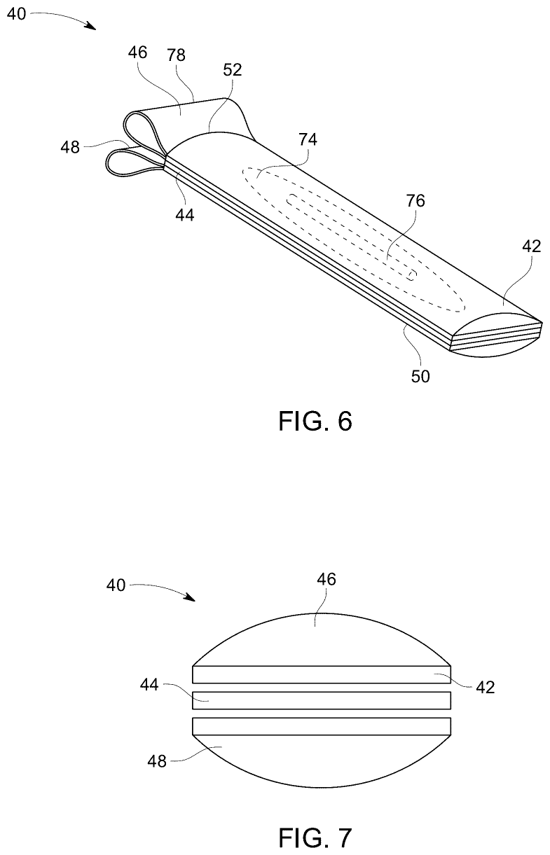

FIG. 6 is a perspective illustration of a bacteria-proof packaging container 40, in accordance with embodiments of the present disclosure. FIG. 7 is an end view illustration of the bacteria-proof packaging container of FIG. 6, in accordance with this example of the present disclosure. With reference to FIGS. 6 and 7, the bacteria-proof packaging container 40, which may be referred to as “packaging 40” includes a bacteria-proof packaging container having a first portion 42 and a second portion 50. The first portion 42 of the bacteria-proof packaging container may be sealed to the second portion 50 of the bacteria-proof packaging container. The seal, which may be formed adhesively between the first portion 42 and second portion 50, creates an interior compartment 74 between the first portion and the second portion. The first portion may be separatable from the second portion along a vertical separation structure 44. A medical implement 76 may be positioned within the interior compartment 74. The medical implement 76 is exemplary, in that the medical implement may be any type of sterile medical implement, including for example a swab, a wipe, a syringe, or a surgical instrument. One skilled in the art would recognize that more than one medical implement may be contained within a single interior compartment 74. The first portion 42 may have a first flap 46 and the second portion may have a second flap 48. The first flap and the second flap may be graspable by a medical professional in preparation to unseal or separate the first portion from the second portion along the vertical separation structure. The first portion 42 may have a first pocket 52 positioned proximate to a top edge 78 of the packaging 40.

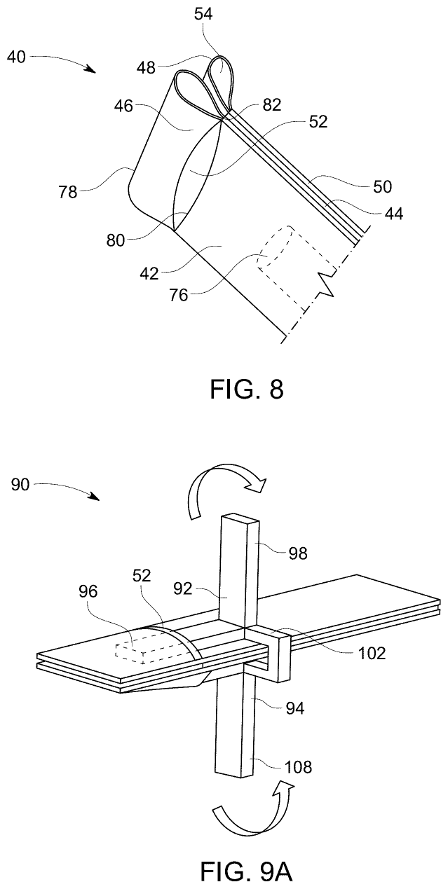

FIG. 8 is another perspective illustration of packaging 40. In FIG. 8, the first portion 42 may be sealed to a second portion along a vertical separation structure 44. The seal joining the first portion 42 to a second portion 50 may have a top seam 80 forming a top of an interior compartment containing medical implement 76. In this example, the first portion 42 may be overfolded to create first flap 46. The overfolded portion may be connected proximate to lateral edges of first portion 42 forming a lateral seam 82 proximate to the lateral edge of the overfolded portion. Lateral seam 82 may run the entire length of the overfolded portion. In this example, part of the overfolded portion is not sealed proximate to the medical implement 76. The overfolded portion, sealed edges, and open edge form first pocket 52. A second portion 50 may be connected to the first portion 42 and has an overfolded structure to create second flap 48 and second pocket 54.

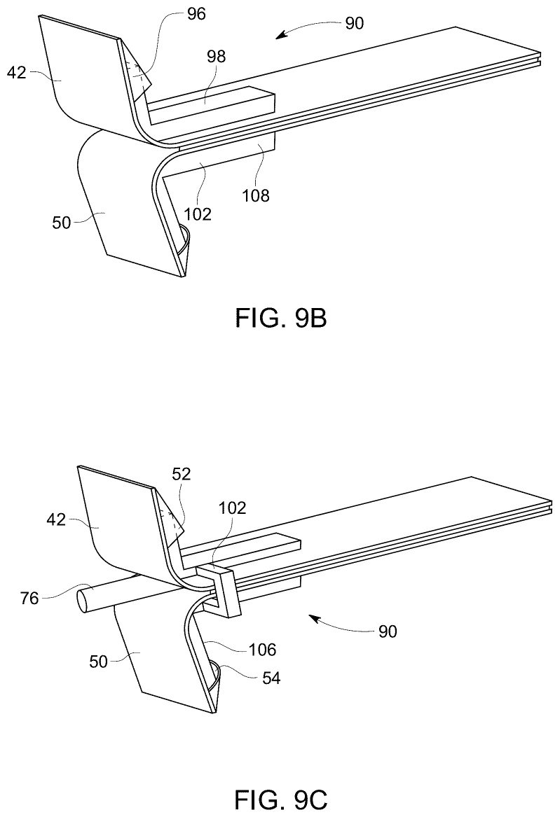

FIGS. 9A-9C are illustrations of a tool 90 interacting with a bacteria-proof packaging container consistent with packaging 40. The tool 90 has a first side 92 and a second side 94 mechanically connected by a member 102 which is positioned at least partially between the first side 92 and the second side 94. The first side 92 of the tool has a first end of the first side 96 and a second end of the first side 98. Similarly, the second side 94 of the tool has a first end of the second side 106 and a second end of the second side 108. The first ends may be shaped to insert into the pockets of packaging 40. The first end of the first side 96 may interact with the first pocket 52 of the first portion 42. The first end of the second side 106 may interact with the second pocket 54 of the second portion 50. First side 92 and second side 94 may be made of metal, plastic, wood, cardboard, or any material or combination of materials with sufficient rigidity to separate first portion 42 from second portion 50. The member 102 that connects the first side 92 to the second side 94 may be flexible, rigid, or a combination thereof. The member 102 may connect to the first side 92 and the second side 94 in any number of ways, including with hinges or elastomeric connections, combinations thereof, or any other mechanical connections. For instance, it may be possible to form member 102 from a plastic material which has a living hinge formed therein, such that portions of the member 102 actuate about the living hinge when the first side 92 and second side 94 are biased towards or away from each other. Member 102 may also disconnect the first side 92 from the second side 94 upon actuation of the tool 90. Disconnection of member 102 upon actuation may offer an advantage in that it may make the tool 90 single-use. Disconnection of member 102 may also offer manufacturing advantages in the design of the connection of member 102 to the first side 92 and the second side 94. The connection of the first side 92 to the second side 94 by member 102 may offer an advantage to a medical professional who may open sterile or bacteria-proof packaging with only one hand. The member 102 may enable control of relative positions of the first side 92 and the second side 94 to enable the first side and the second side to be inserted within the first pocket 52 and the second pocket 54 using only one hand. Further, the member 102 may enable control of the positions of the first side 92 and the second side 94 during the opening of packaging 40. Thus, the member 102 may be configured to enable a medical professional to control the first side 92 and the second side 94 of tool 90 to separate the first portion 42 from the second portion 50 of the bacteria-proof packaging container 40, for example simultaneously, or by use of only one hand.

In operation, tool 90 may be positioned on either side of a medical implement 76 within a bacteria-proof packaging container. The first end of the first side 96 may be inserted in the first pocket 52 and the first end of the second side 106 may be inserted in the second pocket 54. FIG. 9A shows the tool 90 inserted with the packaging closed or sealed. To open the bacteria-proof packaging container, the second end of the first side 98 and the second end of the second side 108 may be moved toward each other, pulling on the first pocket 52 and the second pocket 54, thereby separating the first portion 42 from the second portion 50 along vertical separation structure 44. FIG. 9B shows the packaging after the tool was actuated to open the packaging 40 by separating the first portion 42 from the second portion 50. FIG. 9C shows the system after separating the first portion 42 from the second portion 50, where medical implement 76 may be exposed and manipulable by a medical professional.

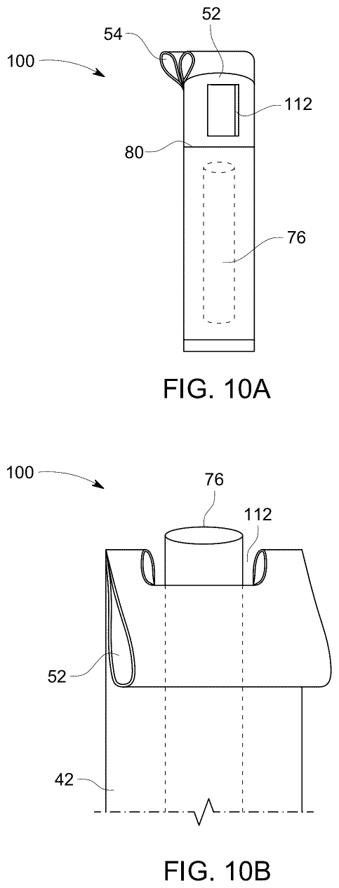

FIGS. 10A-10B are illustrations of the bacteria-proof packaging container 100 consistent with an example of the present invention. In this example, the packaging container 100 has a hole 112 between top seam 80 and pocket 54. Hole 112 may be sized to accommodate medical implement 76. A closed packaging container 100 with hole 112 is shown in FIG. 10A. In FIG. 10B, first portion 42 may be separated from second portion by peeling first pocket 52 away from second pocket 54 to expose medical implement 76 in the packaging. This design may be advantageous, in that, after the first portion of the bacteria-proof packaging container is separated from the second portion of the bacteria-proof packaging container, hole 112 allows the separated portions to lay flat so the medical professional may manipulate the medical implement 76 while it is still in the packaging, allowing the medical implement 76 to remain in the sterile packaging while the medical professional prepares to use it on a patient.

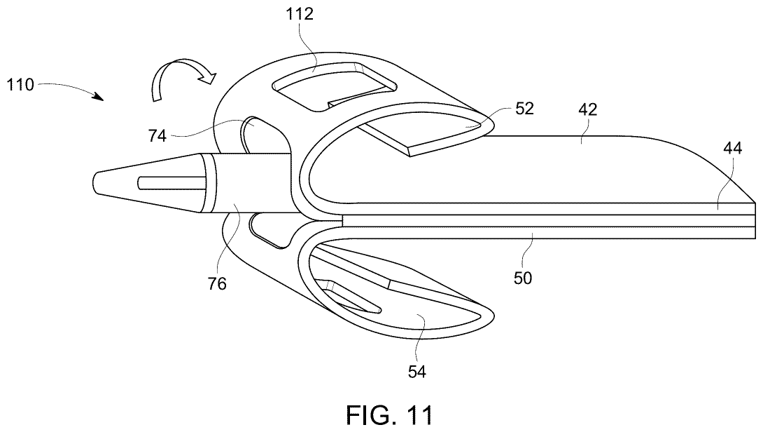

FIG. 11 is an illustration of a bacteria-proof packaging container 110, in accordance with another example of the present disclosure. FIG. 11 shows a bacteria-proof packaging container after separation of a first portion 42 from a second portion 50 along a vertical separation structure 44. The separation may have been effected by moving first pocket 52 away from second pocket 54. In FIG. 11 the medical implement 76 is represented as a sterile syringe with a cap protecting the needle of the syringe. A hole 112 may be sized to accommodate the syringe, allowing the medical professional to firmly grasp the syringe and the syringe to remain in the sterile packaging while the cap is removed from the needle.

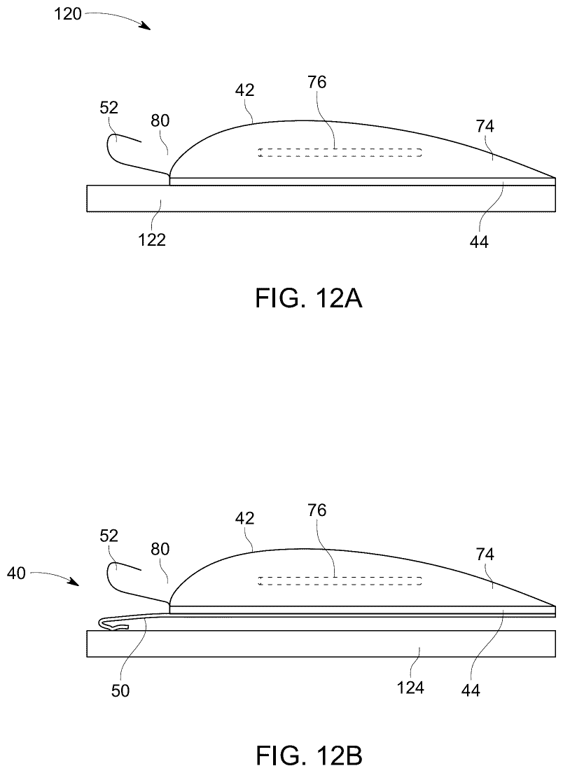

FIGS. 12A and 12B are illustrations of a packaging 120 and a packaging 40, in accordance with additional examples of the present disclosure. In FIG. 12A, a first portion 42 of the packaging 120 may be connected to a rigid second portion 122. A medical implement 76 may be positioned within an interior chamber 74 formed between the first portion 42 and the second portion 122. A pocket 52 may be formed from an overfolded section of the first portion positioned above top seam 80. Thus, the packaging 120 may be opened along vertical separation structure 44 by separating the first portion 42 from the rigid second portion 122 by pulling pocket 52. In the example shown in FIG. 12B, a first portion 42 of the packaging 40 may be connected to second portion 50, which may be positioned proximate to a rigid surface 124. A medical implement 76 may be positioned within an interior chamber 74 formed between the first portion 42 and the second portion 122. A pocket 52 may be formed from an overfolded section of the first portion positioned above top seam 80. A user may apply pressure to at least one of the medical implement 76 or the packaging 40 to create pressure that allows pocket 52 to be pulled away from the second portion 50. Second portion 50 may be flexible or rigid or any combination thereof. Thus, the packaging 120 may be opened along vertical separation structure 44 by separating the first portion 42 from the second portion 50 positioned proximate to the rigid surface 124 by pulling pocket 52 away from the rigid surface 124.

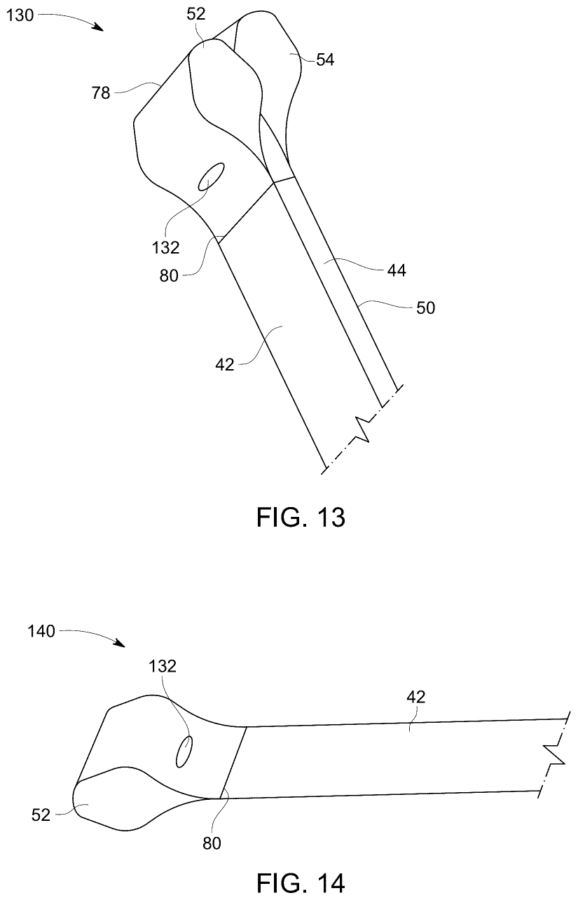

FIG. 13 is an illustration of a packaging 130, in accordance with another example of the present disclosure. In packaging 130, as in packaging 40, first portion 42 may be connected to second portion 50 along vertical separation structure 44. However, in packaging 130 the overfolded portion of the first portion 42 may be folded toward the second portion 50, forming a pocket 52 between the first portion and the second portion instead of on the outside of the first portion. Similarly, second pocket 54 is also formed between the first portion 42 and the second portion 50. In this example, access to the pocket may be through hole 132 formed in first portion 42 between top seam 80 and top edge 78. Positioning hole 132 between top seam 80 and top edge 78 may be advantageous to retain sterility of a medical implement positioned within the bacteria-proof packaging container. A similarly positioned hole in the second portion 50 may allow access to pocket 54. This design may have manufacturing advantages, in that adhesive applied to the packaging to form first pocket 52 and second pocket 54 may need to be only on one side of the packaging. Overfolding ends that have adhesive applied on only one side to form a pocket may be less expensive to manufacture than applying adhesive on more than one side of packaging to form a pocket.

FIG. 14 is an illustration of a packaging 140, in accordance with an example of the present disclosure. In packaging 140, as in packaging 40, pocket 52 may be formed from an overfolded part of first portion 42. However, in packaging 140 the overfolded portion of the first portion 42 may be sealed proximate to top seam 80. Access to pocket 52 may be through hole 132 in the overfolded part of portion 42.

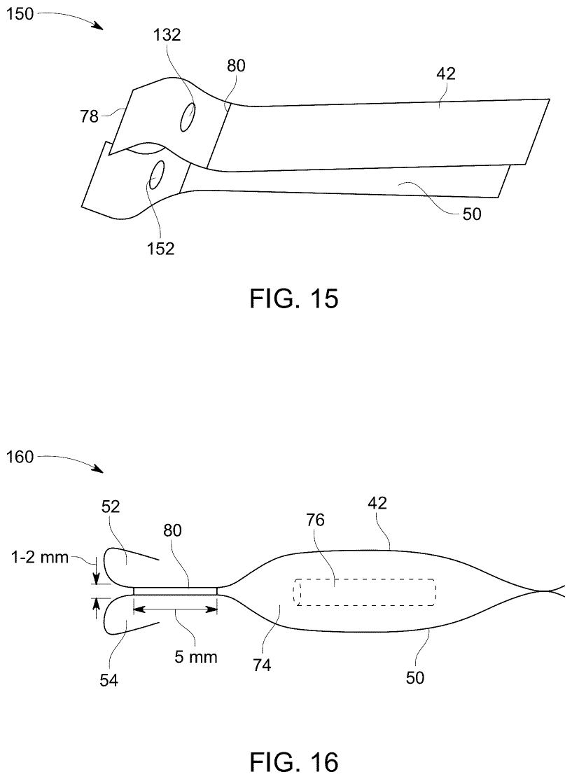

In reference to FIG. 15 and FIGS. 9A-9C, FIG. 15 has an illustration of a packaging 150, in accordance with yet another example of the present disclosure. In packaging 150, as in packaging 40, first portion 42 is connected to second portion 50. However, in packaging 150 the first end of the first side 96 of tool 90 (from FIGS. 9A-9C) may be inserted through hole 132 in the first portion 42 positioned between top seam 80 and top edge 78. The first end of the second side 106 of tool 90 may be inserted through hole 152 in the second side of the second portion 50. The tool would interact to open packaging 150 by pulling on the interiors of the first portion 42 and the second portion 50 instead of being inserted into and interacting with pockets. This design of the invention may have manufacturing advantages, as not requiring an overfold may have comparatively fewer steps in the manufacturing process, and therefore be less expensive to manufacture than an overfolded design.

FIG. 16 is an illustration of a packaging 160, in accordance with another example of the present disclosure. In packaging 160, as in packaging 40, first portion 42 may be connected to second portion 50 forming interior chamber 74. Medical implement 76 may be positioned within interior chamber 74. In packaging 160 top seam 80 has dimensions of about 5 millimeters (mm). First pocket 52 may be positioned about 2 millimeters from second pocket 54.

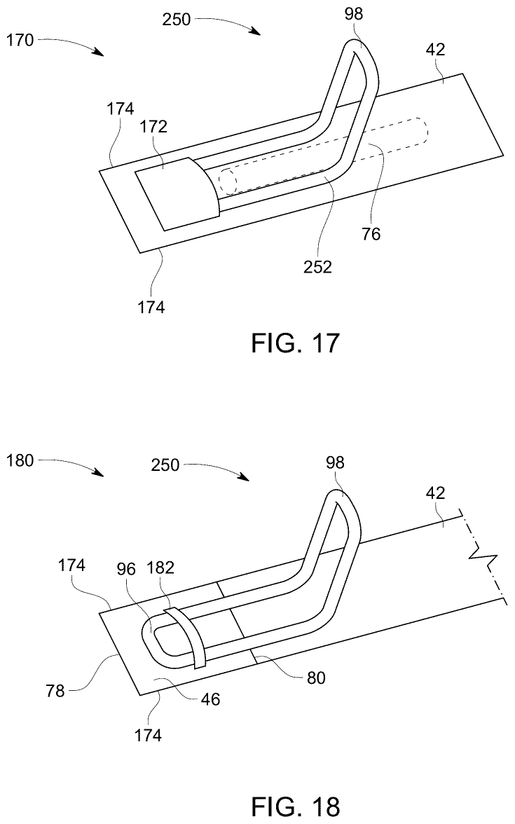

FIG. 17 is an illustration of a packaging 170, in accordance with another example of the present disclosure. In packaging 170, tool 250 may be inserted into a pocket formed by a piece 172 that may be connected proximate to lateral edges 174 of first portion 42. Tool 250 may accommodate medical implement 76 positioned within the packaging. Second end of first side 98 of tool 250 may be positioned above medical implement 76. The tool 250 may be controllable to move the second end of first side 98 of tool 250 toward the medical implement 76, exerting pressure on piece 172, and pulling on first portion 42. The movement of tool 250 may be a rocking motion, a rotation, or any combination of motions that pulls the first portion 42. Piece 172 may be made from the same material as first portion 42 or from any number of different materials or combination of materials that may be connected to first portion 42. The first end of first side 96 (FIG. 18) of tool 250 may be connected to the second end of first side 98 by at least one corner 252 that may be rounded, smooth, or angular or any combination thereof. A rounded or smooth corner 252 may be preferred to facilitate motion of the second end of the first side 98.

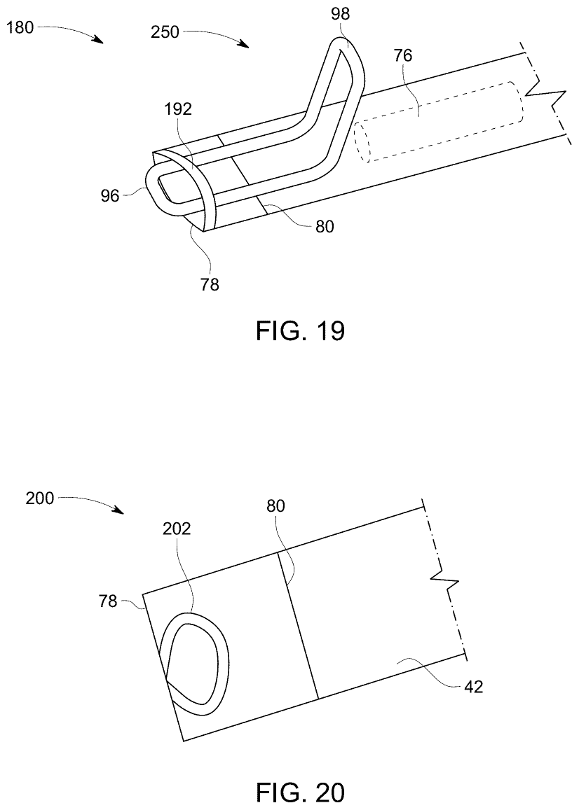

FIGS. 18 and 19 are illustrations of a packaging 180, in accordance with an example of the present disclosure. In packaging 180, tool 250 may be inserted into a pocket formed by a band 182. The band 182 may be made of the same or different material as the first portion 42. Band 182 may be a thinner version of piece 172. Band 182 may be connected on lateral edges 174 of first portion 42 or may pierce first portion 42 and may terminate or connect to itself between first portion 42 and second portion 50. First end of first side 96 of tool 250 may be sized to be positioned within the band. When second end of first side 98 of tool 250 is moved toward the medical implement 76, tool 250 exerts pressure on band 182, pulling on first portion 42 and opening the sterile package. In FIG. 18, the band 182 may be positioned between the top edge 78 and the top seam 80, centrally on first flap 46. In FIG. 19, the band 192 may be positioned proximate to top edge 78. The first end of first side 96 of tool 250 may extend proximate to or beyond top edge 78. Extension of tool 250 beyond top edge 78 may offer advantages in that it may allow the medical practitioner flexibility to open less than all of the packaging 40. By extending the tool 250 beyond edge 78, less of first portion 42 will be separated from second portion 50, which may thereby expose less than all of the medical implement 76. The open design proximate to top edge 78 may allow the medical practitioner to customize the portion of packaging opened.

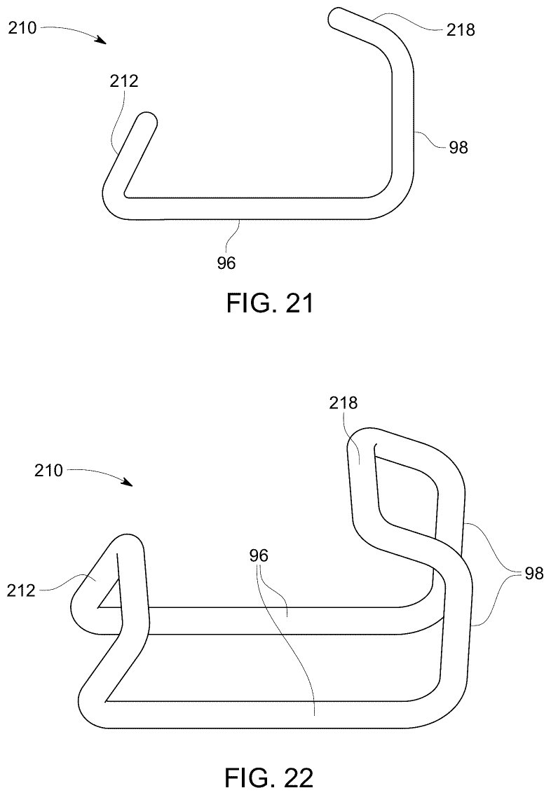

FIG. 20 is an illustration of a packaging 200, in accordance with yet another example of the present disclosure. In packaging 200, tool 250 may be inserted into a pocket formed by a loop 202. The loop 202 may be made of the same or different material as the first portion 42. Loop 202 may be connected centrally proximate to top edge 78 of first portion 42. Loop 202 may be sized to accommodate first end of first side 96 of tool 250 (FIGS. 17-19) or tool 210 (FIGS. 21-22).

Packaging 180 and 200 may optimally interact with a tool 210 with a hook 212 proximate to first end of first side 96 and directed back toward the second end of first side 98 of a tool 210. FIGS. 21 and 22 are illustrations of a tool 210, in accordance with examples of the present disclosure. FIG. 21 is a side profile view of tool 210 showing the first end of first side 96 directed back toward second end of first side 98 forming the hook of first side 212. Second end of first side 98 may be additionally shaped to accommodate a medical implement within the packaging by forming a portion of second end of first side 98 such that when second end of first side 98 is moved toward a medical implement, first shaped end 218 may fit around the medical implement, allowing the tool 210 to open the bacterial-proof packaging container to a larger degree than a tool without first shaped end 218. FIG. 22 is a perspective view of tool 210 showing an accommodation to fit around a medical implement. Tool 210 may be made of metal, plastic, wood, cardboard, or any material or combination of materials of suitable rigidity that can supply sufficient pressure to open packaging 180, 190, or 200.

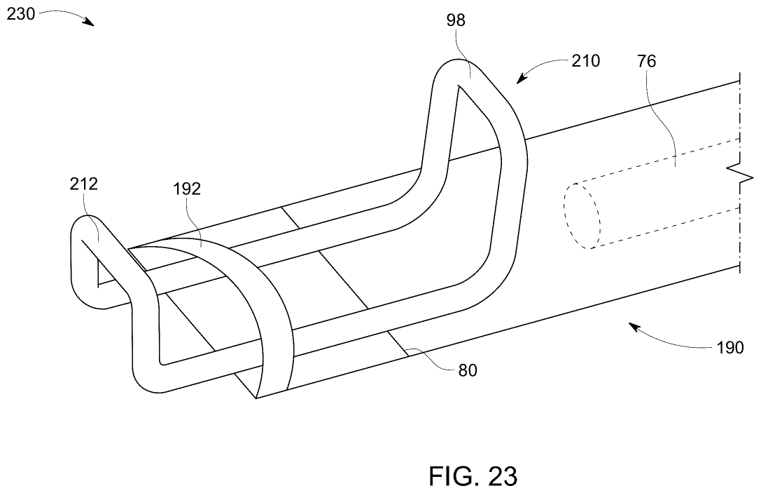

FIG. 23 is an illustration of a system 230, in accordance with examples of the present disclosure. In system 230, hook 212 of tool 210 (FIGS. 21-22) catches band 192 of packaging 190. When second end of first side 98 is moved toward medical implement 76, tool 210 exerts pressure on band 192, opening packaging 190.

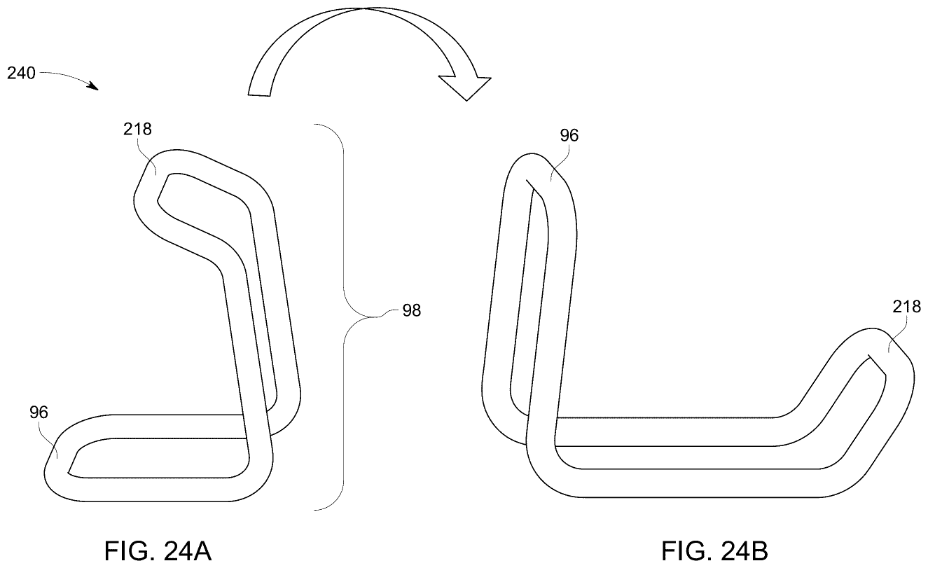

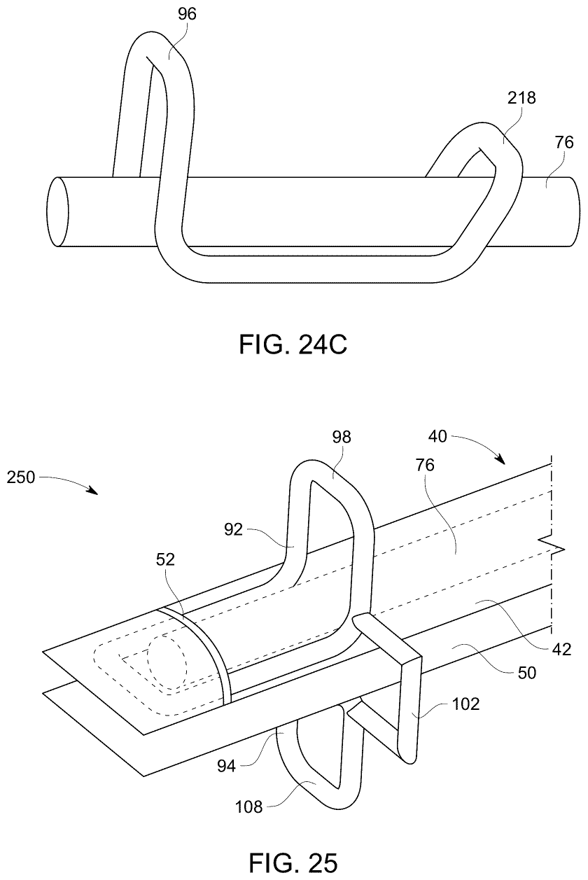

FIGS. 24A-24C are illustrations of a tool 240, in accordance with additional examples of the present disclosure. Tool 240 has a first end of first side 96 and a second end of first side 98 that are positioned angularly to each other. Second end of first side 98 may optimally have a first shaped end 218 that may be sized to accommodate a medical implement 76 positioned within a bacteria-proof packaging container. FIG. 24A shows the tool 240, which when rotated may be the tool 240 in FIG. 24B. FIG. 24C shows the tool 240 in a position similar to FIG. 24B and illustrates how the first shaped end 218 may accommodate a medical implement 76 when the tool is positioned with an opened bacteria-proof packaging container.

FIG. 25 is an illustration of a tool 250, in accordance with another example of the present disclosure. In FIG. 25, tool 250 may have a first side 92 and a second side 94. First side 92 has a first end of first side 96 and a second end of first side 98. Second side 94 has a second end of second side 108. The first side 92 and second side 94 may be mechanically connected by member 102. The mechanical connection of member 102 to at least one of the first side and the second side may be moveable to enable motion of a first end and a second end, for example, by a rocking motion. The first end of the first side 96 may be inserted into first pocket 52 of packaging 40. Packaging 40 has a first portion 42 and a second portion 50. Tool 250 may be shaped to accommodate medical implement 76 positioned between first portion 42 and second portion 50.

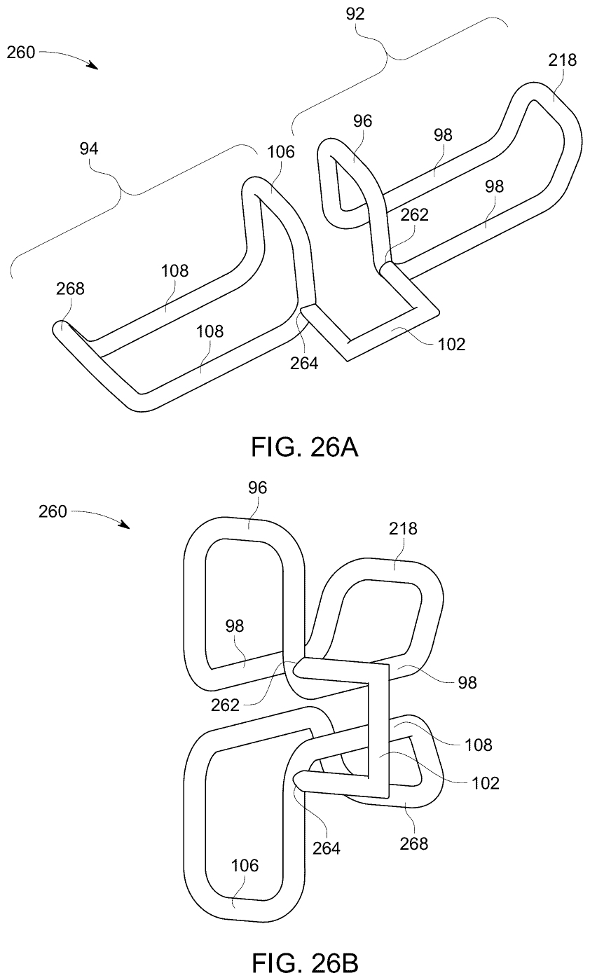

FIGS. 26A and 26B are an illustration of a tool 260, in accordance with preferred examples of the present disclosure. In FIG. 26A, tool 260 has a first side 92 and a second side 94. First side 92 has a first end of first side 96 and a second end of first side 98. Second end of first side 98 has a first shaped end 218. Second side 94 has a first end of second side 106 and a second end of second side 108. Second end of second side 108 may further have a second shaped end 268. Member 102 connects first side 92 to second side 94 at first connection 262 and second connection 264. In FIG. 26A, tool 260 may be in its closed position, where first end of first side 96 and first end of second side 106 may be inserted into pockets of packaging. In FIG. 26B, the tool may be in its open position, where second end of first side 98 and second end of second side 108 may have been moved toward each other. This position of the second ends of tool 260 may move the first end of first side 96 and first end of second side 106 away from each other. This movement of first ends of the tool 260 away from each other while the first ends are inserted into pockets may open the packaging. Member 102 may keep first side 92 connected to second side 94 at first connection 262 and second connection 264. The connections may be any connection that allows the motion of the tool, for example elastomeric connections or hinges. Member 102 may also disconnect first side 92 from second side 94 when the tool is actuated. When second end of first side 98 is positioned close to second end of second side 108 in tool 260 open position, the first shaped end 218 and second shaped end 268 may be positioned around a medical implement.

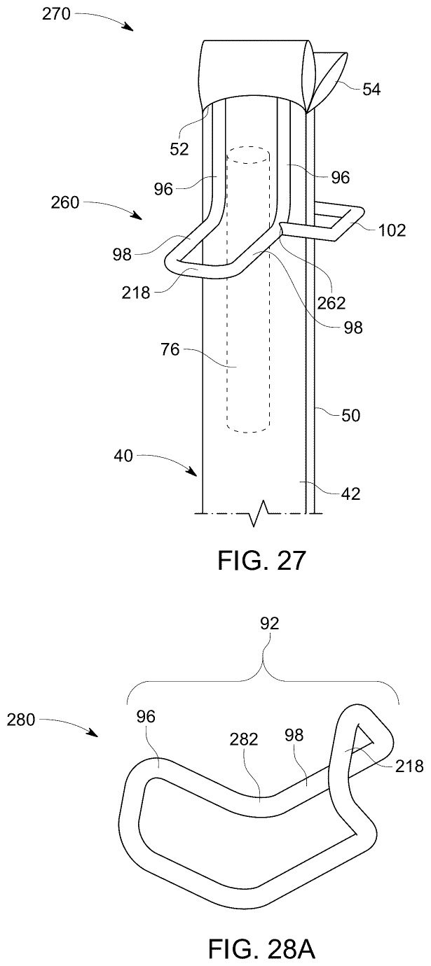

FIG. 27 is an illustration of a system 270, in accordance with another example of the present disclosure. In FIG. 27, first end of first side 96 of tool 260 may be inserted into pocket 52 formed from the first portion 42 of packaging 40. Member 102 connects the first side 92 of tool 260 to the second side 94 (FIG. 26A) of tool 260 at first connection 262. The first end of the second side 106 (FIG. 26A) of tool 260 may be inserted into pocket 54 formed from the second portion 50 of package 40. Medical implement 76 may be positioned between first portion 42 and second portion 50. Tool 260 may be positioned around medical implement 76. First shaped end 218 may accommodate medical implement 76 when tool 260 is moved into an open position by actuating the tool to move second end of first side 98 toward the second end of the second side 108 (FIG. 26B) to open packaging 40 by moving first pocket 52 away from second pocket 54 using the inserted first ends of tool 260.

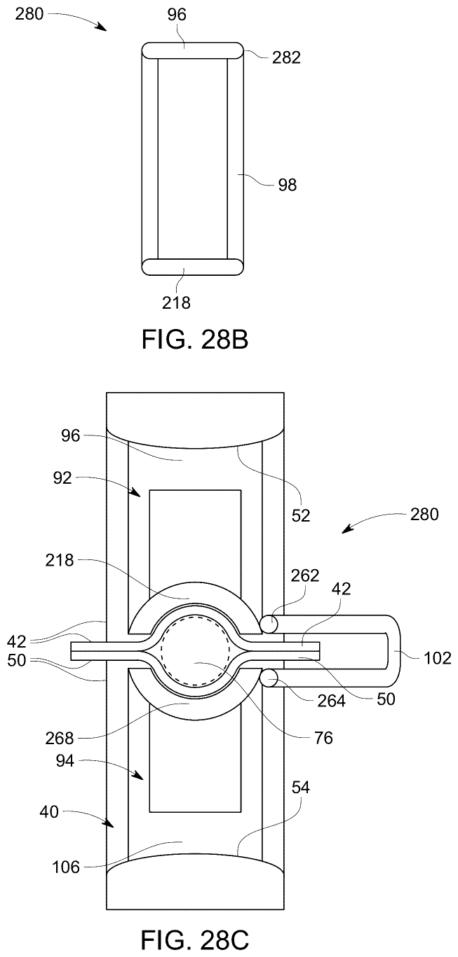

FIGS. 28A-28C are illustrations of a tool 280, in accordance with examples of the present disclosure. FIG. 28A shows a perspective view of a first side 92 of tool 280, which has a first end of first side 96, a second end of first side 98. The first end of first side 96 and second end of first side 98 may be positioned at an angle 282 to each other. Second end of first side 98 may have a first shaped end 218. FIG. 28B shows the top view of the first side of tool 280 where the angle between the first end of first side 96 and the second end of first side 98 is a right angle. The angle may be a curve, a right angle, or any angle that allows the tool 280 to be actuated to open packaging 40. FIG. 28C shows an end-on view of the tool 280 with opened packaging 40. FIG. 28C shows a first side 92 of tool 280 connected to a second side 94 by member 102. Member 102 connects to first side 92 through first connection 262. Member 102 connects to second side 94 through second connection 264. First side 92 may be positioned on top of first portion 42 of packaging 40. First end of first side 96 may be positioned within first pocket 52 of first portion 42 of packaging 40. Second side 94 may be positioned below second portion 50 of packaging 40. Medical implement 76 may be between first portion 42 and second portion 50. First end of second side 106 may be positioned within second pocket 54 of second portion 50. In a preferred configuration, first shaped end 218 and second shaped end 268 may accommodate medical implement 76 when tool 280 may be in the open position, which may allow first end of first side 96 and first end of second side 106 to move farther away from each other than if first shaped end 218 and second shaped end 268 do not accommodate medical implement 76.

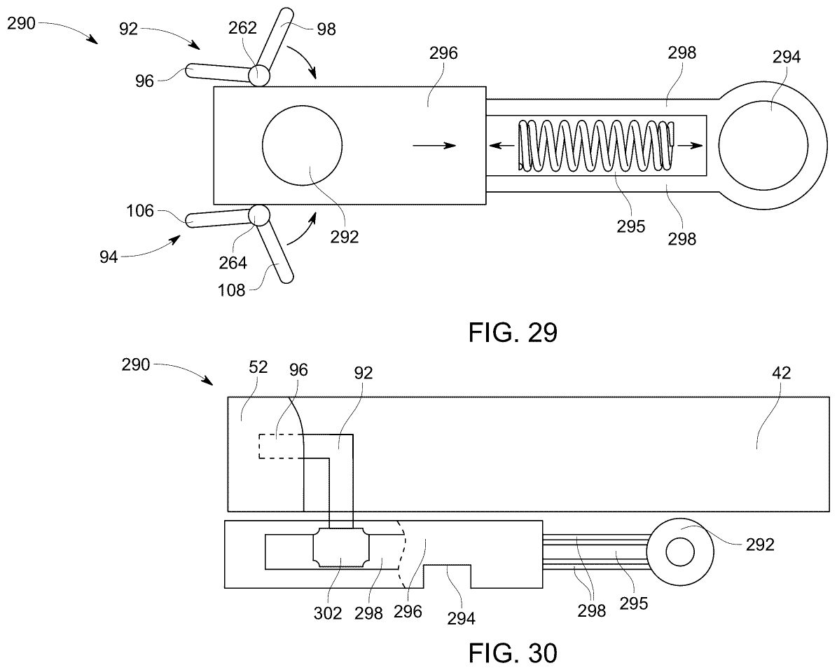

FIGS. 29 and 30 are illustrations of a tool 290, in accordance with additional examples of the present disclosure. Tool 290 has a first side 92 and a second side 94. First side 92 has a first end of the first side 96 and a second end of the first side 98. First side 92 is connected to housing 296 by first connection 262. Housing 296 is connected to second side 94 through second connection 264. Housing 296 may have a first contact structure. The first contact structure 292 may be sized to accommodate a first finger. Second side 94 has a first end of the second side 106 and a second end of the second side 108. Housing 296 may have an insert 298 that may be slidably positionable relative to the housing 296. Insert 298 may be operably connected to the first side 92 and the second side 94. Insert 298 may have a second contact structure 294 that may be sized to accommodate a second finger. Insert 298 also may contain a bias mechanism 295 that may be positioned between the insert 298 and the housing 296. The bias mechanism may bias the first contact structure 292 away from the second contact structure 294. Bias mechanism 295 may be a spring, elastomer, or any other structure that may bias the first contact structure 292 away from the second contact structure 294. Moving the insert 298 towards the housing 296 may cause the first end of the first side 96 and the first end of the second side 106 to move away from each other. FIG. 29 is a side view of tool 290. FIG. 30 is a top view of tool 290 and shows first end of first side 96 inserted into pocket 52 of the first portion 42 of packaging 40. In FIG. 30, housing 296 may be cut away to show an example of an operable connection 302 of the first side 92 to the insert 298.

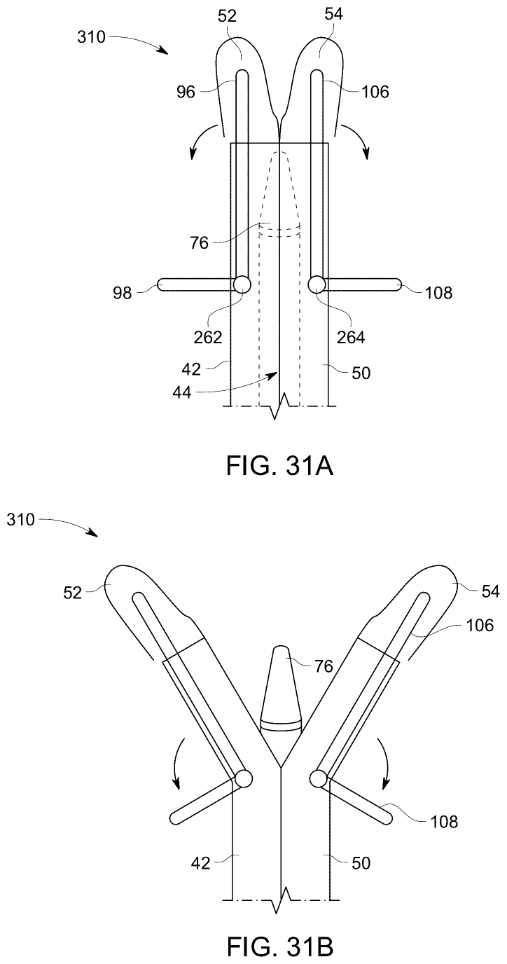

FIGS. 31A and 31B are illustrations of a system 310, in accordance with examples of the present disclosure. In system 310, tool 290 (FIG. 29) interacts with packaging 40. FIG. 31A shows the tool 290 prepared to open the packaging 40. FIG. 31B shows tool 290 opening packaging 40. FIG. 31A shows a medical implement 76 positioned within packaging 40. Packaging 40 may have a first portion 42 and a second portion 50 connected by separation structure 44. First portion 42 may have a pocket 52. Similarly, second portion 50 may have a second pocket 54. First end of the first side 96 of tool 290 may be positioned within pocket 52. Second end of first side 98 of tool 290 may be positioned at an angle to first end of first side 96. First end of second side 106 of tool 290 may be positioned inside pocket 54. Second end of second side 108 may be positioned at an angle to first end of second side 106. When the tool 290 is actuated, first end of first side 96 may move away from first end of second side 106 and second end of first side 98 may move toward second end of second side 108. First end of first side 96 may move away from first end of second side 106, pulling pocket 52 to open packaging 40. Similarly, first end of second side 106 may move away from first end of first side 96, pulling pocket 54 to open packaging 40. FIG. 31B shows a partially opened packaging 40 with pockets 52 and 54 separated from each other to expose medical implement 76.

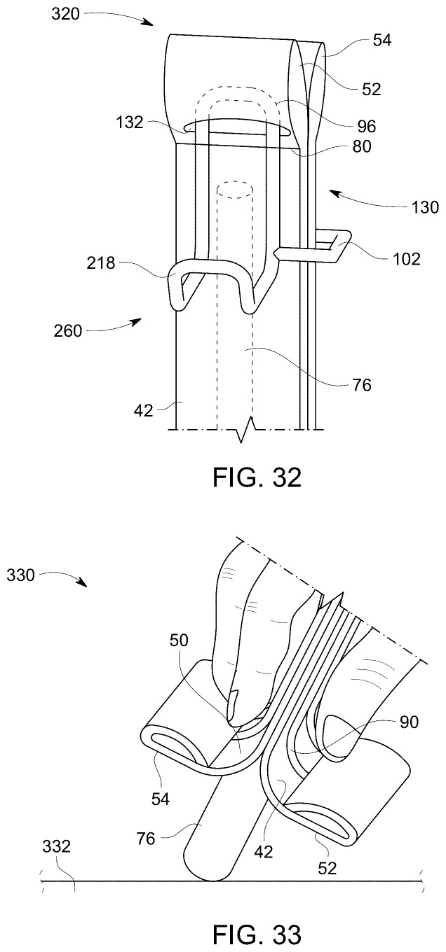

FIG. 32 is an illustration of a system 320, in accordance with an example of the present disclosure. In system 320, tool 260 may interact with packaging 130. Medical implement 76 may be positioned between the first portion 42 and the second portion 50 (FIG. 13) of packaging 130. First end of the first side 96 of tool 260 may access pocket 52 through hole 132. Member 102 may connect the first side 92 (FIG. 26A) of tool 260 to the second side 94 (FIG. 26A) of tool 260. The first end of the second side 106 (FIG. 26A) of tool 260 may similarly access pocket 54 through a hole in the pocket. When the tool is actuated to move first shaped end 218 toward medical implement 76, the package may open and first shaped end 218 may position around medical implement 76.

FIG. 33 is an illustration of a medical professional using a system 330 with tool 90 and packaging 40 in another example of the present disclosure. In this example, tool 90 may be used to open packaging 40 and apply the medical implement 76, which may be an antiseptic wipe, to a patient's skin 332 while the medical implement may be partially in packaging 40. In FIG. 33, the medical professional may have used one hand to actuate tool 90 to pull pocket 52 of first portion 42 away from pocket 54 of second portion 50. The at least partially opened package exposes the antiseptic wipe, which may have an unexposed portion remaining in the packaging 40. The medical professional may grasp the medical implement through the packaging while the tool remains in contact with the packaging. The medical professional can then apply the medical implement 76 to a patient without having to touch the medical implement, thereby limiting exposure of the medical professional to an antiseptic solution on the sterile wipe. The tool 90 may hold the packaging 40 at least partially open while the medical professional applies the medical implement 76 to the patient's skin 332.

The one-handed operation of the system 330 to open a bacteria-proof packaging container 40 may offer several advantages over conventional sterile packaging systems. For example, one-handed operation of the system 330 may increase efficiency in the medical care setting by freeing a medical professional's second hand to perform any number of other tasks, including continuing patient care. A second example of an advantage of one-handed operation of the system is facilitating the opening of a bacteria-proof packaging container by a physically disabled medical professional, thereby increasing the number of medical professionals available to provide patient care. Though one-handed operation may provide certain advantages, two hands may be also used to operate the system 330 and still retain the advantage of limiting exposure of the medical implement to the medical professional and the advantage of holding the packaging open to expose the medical implement.

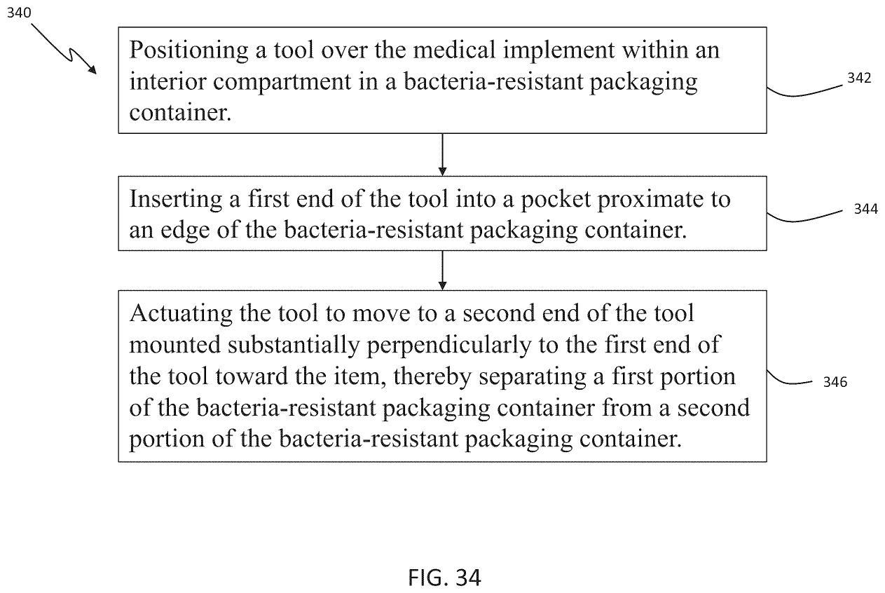

FIG. 34 is a flowchart 340 illustrating a method to open a package while maintaining sterility of a medical implement within the package in accordance with an example of the disclosure. It should be noted that any process descriptions or blocks in flow charts should be understood as representing modules, segments, or steps that include one or more instructions for implementing specific logical functions in the process, and alternate implementations are included within the scope of the present disclosure in which functions may be executed out of order from that shown or discussed, including substantially concurrently or in reverse order, depending on the functionality involved, as would be understood by those reasonably skilled in the art of the present disclosure.

Step 342 includes positioning a tool over the medical implement within an interior compartment in a bacteria-resistant packaging container.

Step 344 includes inserting a first end of the tool into a pocket proximate to an edge of the bacteria-resistant packaging container.

Step 346 includes actuating the tool to move to a second end of the tool mounted substantially perpendicularly to the first end of the tool toward the item, thereby separating a first portion of the bacteria-resistant packaging container from a second portion of the bacteria-resistant packaging container. The packaging container may be torn, pulled apart, peeled, or otherwise separated to expose the medical implement. In one example, the opened packaging container may expose less than all of the medical implement. This may allow a user to grip the medical implement while using or manipulating the medical implement. The unexposed end may still be covered by a portion of the packaging container, thereby retaining sterility, and allowing manipulation of the medical implement. A medical professional may apply the medical implement to the patient without removing the medical implement from the packaging, thereby limiting exposure of the medical professional to the medical implement.

Any number of additional steps, functions, processes, or variants thereof may be included in the method, including any disclosed relative to any other figure of this disclosure.

It should be emphasized that the above-described embodiments of the present disclosure, particularly, any “preferred” embodiments, are merely possible examples of implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure and the present disclosure and protected by the following claims.

What is claimed is:

1. A system for opening a package while maintaining sterility of a medical implement within the package, comprising:
a bacteria-proof packaging container having an interior compartment;
a first portion of the bacteria-proof packaging container separatable from a second portion of the bacteria-proof packaging container;
a medical implement positioned within the interior compartment;
at least one pocket positioned proximate to a top edge of the bacteria-proof packaging container; and
a tool configured for opening the bacteria-proof packaging container, wherein the tool has at least one end sized to fit within the at least one pocket, and wherein a first end of the tool is angularly connected to a second end of the tool, wherein the tool is controllable to separate the first portion from the second portion.

2. The system of claim 1, wherein the bacteria-proof packaging container further comprises a vertical separation structure positioned between the first portion and the second portion, and axially along the bacteria-proof packaging container, wherein pulling the at least one pocket by the tool causes the bacteria-proof packaging container to open along the vertical separation structure to separate the first portion from the second portion.

3. The system of claim 1, wherein the tool further comprises:
a first side and a second side, wherein the first and second sides are positionable on opposite sides of the medical implement;
a member joining the first side to the second side; and
wherein the at least one pocket further comprises first and second pockets positioned on the first and second portions, respectively, wherein each of the first and second pockets are positioned proximate to the top edge of the bacteria-proof packaging container, and wherein the first and second pockets are configured to receive the first and second sides of the tool, respectively, and wherein when the first side and the second side are positioned in the first and second pockets, respectively, the first side and second side are moveable away from each other to separate the first portion from the second portion.

4. The system of claim 1, wherein the tool is sized to separate a portion of the bacteria-proof packaging container to expose the medical implement in the interior compartment.

5. The system of claim 1, wherein the second portion of the bacteria-proof packaging container is rigid.

6. The system of claim 1, wherein the at least one pocket further comprises an overfolded section of the first portion connected proximate to lateral edges thereof.

7. The system of claim 1, wherein the at least one pocket further comprises a piece, wherein the piece is connected proximate to lateral edges of the packaging.

8. The system of claim 1, wherein the at least one pocket further comprises a loop connected proximate to the top edge of the bacteria-proof packaging container.

9. The system of claim 8, wherein the tool is positionable through the loop and proximate the top edge of the bacteria-proof packaging container.

10. The system of claim 1, wherein the first end of the tool further comprises a hook.

11. The system of claim 1, wherein the second end of the tool further comprises a shaped end, and wherein the shaped end is sized to accommodate the medical implement.

12. The system of claim 1, wherein the tool further comprises a corner connected between the first side and the second side, and wherein the corner is rounded to facilitate a rocking motion of the tool to open the bacteria-proof packaging container.

13. The system of claim 3, wherein the member is mechanically connected to at least one of the first side and the second side.

14. The system of claim 3, wherein the member is detachably connected to at least one of the first side and the second side.

15. The system of claim 3, the member further comprising:
a housing;
a first contact structure on housing, the first contact structure sized to accommodate a first finger;
an insert slideably positionable relative to the housing and operably connected to the first side and the second side, the insert having a second contact structure sized to accommodate a second finger; and
a bias mechanism positioned between the insert and the housing, the bias mechanism biasing the first contact structure away from the second contact structure, wherein moving the insert towards the housing causes the first side and the second side to move away from each other.

16. A packaging container for maintaining sterility of a medical implement within the package during unsealing, comprising:
a first portion of the bacteria-proof packaging container separatable from a second portion of the bacteria-proof packaging container;
an interior compartment between the first portion and the second portion;
a medical implement positioned within the interior compartment;
at least one pocket positioned proximate to a top edge of the bacteria-proof packaging container, wherein the at least one pocket is formed from an overfolded section connected along lateral edges thereof to the first portion, wherein the at least one pocket is located interior of the overfolded section and the lateral edges.

17. The packaging container of claim 16, wherein the at least one pocket further comprises first and second pockets positioned on the first and second portions, respectively, wherein each of the first and second pockets are positioned proximate to the top edge of the bacteria-proof packaging container, and wherein the first and second pockets are configured to receive the first and second sides of the tool, respectively, and wherein when the first side and the second side are positioned in the first and second pockets, respectively, the first side and second side are moveable away from each other to separate the first portion from the second portion.

18. A method to open a package while maintaining sterility of a medical implement within the package, comprising:
positioning a tool over the medical implement within an interior compartment in a bacteria-resistant packaging container;
inserting a first end of the tool into a pocket proximate to a top edge of the bacteria-resistant packaging container; and
actuating the tool to move to a second end of the tool mounted substantially perpendicularly to the first end of the tool toward the item, thereby separating a first portion of the bacteria-resistant packaging container from a second portion of the bacteria-resistant packaging container.

19. The method of claim 18, further comprising:
positioning the bacteria-proof packaging container proximate to a rigid surface; and
using the tool to pull the pocket away from the rigid surface, thereby separating the first portion of the bacteria-proof packaging container from the second portion of the bacteria-resistant packaging container.

20. The method of claim 18, wherein actuating the tool to move to a second end of the tool toward the item further comprises using a single hand, whereby the tool is held with the single hand and at least one finger of the single hand causes movement of the second end of the tool.
